(19) United States
(12) Patent Application Publication
(10) Pub. No.: US 2008/0207571 A1
(43) Pub. Date: Aug. 28, 2008

(54) Regimens for Oral Monophasic Contraceptives

(75) Inventor: Victoria Jane Davis, Toronto (CA)

Correspondence Address: ORGANON USA, INC.;c/o Schering-Plough Corporation, 2000 Galloping Hill Road, Mail Stop: K-6-1, 1990, Kenilworth, NJ, 07033, US

(73) Assignee: N.V. Organon, Oss (NL)

(21) Appl. No.: 11/917,492

(22) PCT Filed: Jun. 16, 2006
(86) PCT No.: PCT/US2006/023382

§ 371 Date: Dec. 13, 2007

(30) Foreign Application Priority Data
Jun. 21, 2005 (EP) EP05105481.5

(51) Int. Cl. A61K 31/56; G04B 47/00; A61P 15/18

(52) U.S. Cl. 514/170; 368/10

(57) ABSTRACT

The subject invention provides for new regimens for monophasic oral contraceptives.

The present invention relates mainly to the field of female reproductive medicine, and in particular to human female contraception. The present invention relates to new regimens for administration of oral monophasic contraceptive dosage units, e.g. to achieve contraception or to treat and/or prevent other hormone cycle-dependent indications such as dysmenorrhoea, menorrhagia, irregular menstruation, menstrual migraine and premenstrual syndrome (PMS).

It is standard practice in the field of oral contraceptive regimes that the definition of the cycle duration is linked to a fixed number of days and weeks. These kind of regimens evolved due to the need to mimic the menstrual cycle in the development of contraceptives. As a result thereof, women start a new cycle of contraception at a fixed day of a week, e.g. a Sunday, a Monday etc. Another result thereof is that patient packs with identical content and form (such as oral contraceptive strips with 21 or 28 pills) can be produced which in turn ensures economic production methods and helps the user to acquire habits needed for consistent self-administration of the tablets for contraception.

Monophasic oral contraceptive pills or tablets are well known in the art. Examples of commercially available monophasic oral contraceptives are Marvelon®, Mercilon®, Microgynon®, Yasmin®, Alesse®, Minovral®, Ovral®, Cyclen®, Minestrin®, Estrin®, Ortho 1/35®, Ortho 0.5/35®, Brevicon 1/35®, Brevicon 0.5/35®, Micronor®, Demulin®, Select®, Loestrin®, Yaz®, etc. Most monophasic oral contraceptive pills contain both a progestogen and an estrogen and some contain a progestogen only.

The most common regimen for oral monophasic contraceptives is that a woman takes the pill for 21 days, then stops taking the pill for 7 days (or takes 7 placebo pills) and then restarts taking the pill again for 21 days, etc. These moments (stopping the pill and re-starting the pill) that have to be remembered fall on different dates each time. For example, if a woman stopped taking the pill on January 1, then she has to restart the pill on January 8 and stop again on January 29. The pill will then have to be restarted on February 5 and removed again on February 26 etc. etc. It is clear that it is difficult to keep track of these dates. As a result thereof some women forget to start taking the pill after the seven day break in a timely fashion resulting in unwanted pregnancies.

The subject invention now provides for new regimens for oral monophasic contraceptives (and for the administration of oral monophasic contraceptives to treat and/or prevent other hormone cycle-dependent indications), resulting in improved compliance while maintaining contraceptive efficacy. This improved compliance is thus enabled by the functional combination of at least two reservoirs (containing monophasic oral contraceptive dosage units sufficient for any at least two cycles) during at least two cycles. Compliance starts with the administration of the first dosage unit of the second reservoir. Without any at least second reservoir (containing monophasic oral contraceptive dosage units) one would not have to look at compliance. Therefore to obtain the effect of improved compliance, the functional combination of the use of at least two reservoirs is a prerequisite.

The new regimens of the present invention further result in that women will have only 12 periods a year as opposed to thirteen in standard 21/7 regimens.

Oral monophasic pill regimes do not impose the constraints to provide for a cycle of fixed duration. The present invention now exploits this new facility by providing regimens which are not constrained by identical cycles of fixed duration but enable flexible cycle duration. The subject invention has the important advantage to help user habit acquisition, because start and stop of a regimen of the subject invention is enabled on fixed numerical days of the month. Thus, the invention provides for a contraceptive regime with cycles of hormone administration for defined cycle durations, such that the cycle durations vary in order to correspond with the number of days of the calendar month in which the cycle is starting.

Both “month” and “calendar month” as used herein means any month, i.e. January, February, March, April, May, June, July, August, September, October, November, or December.

A “numerical date” as used herein is any existent date of a month. For example, January has 31 numerical dates. January 1, January 2, January 3 etc. etc. February has 28 or 29 numerical dates; March has 31 numerical dates; April has 30 numerical dates, etc.

“Cycle” or “cycle of contraception” as used in the subject invention is the duration of the number of the days of the month in which the cycle is started. During a cycle there is a hormone-taking phase and a hormone-free phase. For example, a cycle which is started in January is 31 days; a cycle which is started in February is 28 or 29 days depending on whether it is a leap year or not; a cycle which is started in March is 31 days; a cycle which is started in April is 30 days, etc, etc. In addition, a cycle of the subject invention is a partial circle of events wherein the hormone levels in a woman increase and decrease due to the use of the oral monophasic contraceptive. In order to complete the circle of events wherein hormone levels increase, decrease, increase again and decrease again, a woman must complete at least two cycles of oral monophasic contraception.

“A dosage unit” as used herein is a pill or a puff (from a spray device).

“Starting” as used herein means administering or spraying or any other form of contraceptive or pharmaceutical administration. For example, pills are administered and sprays are sprayed.

“Stopping” as used herein means ‘not administering’. For example, pills and sprays are stopped, i.e. not administered.

‘A reservoir’ as used herein means a reservoir suitable to hold an oral contraceptive such as, but not limited to, a bottle of monophasic oral contraceptive pills, a spray device comprising a contraceptive (oral or transdermal) spray, a box containing pills, or a dispenser containing pills.

As used herein a bottle containing contraceptive monophasic pills means just that: a bottle with pills. In the subject invention it is no longer necessary to provide female patients with standard strips of 21 or 28 contraceptive pills but rather just with a bottle with any number of pills. This renders the need to produce blisters and strips for monophasic oral contraceptive products obsolete. A bottle of monophasic oral contraceptive pills can also be a box with pills or a dispenser with pills or any other type of reservoir suitable to hold pills.

“Sufficient” as used in the phrase “a reservoir containing monophasic oral contraceptive dosage units sufficient for at least any ______ cycles of contraception” means that the reservoir has to contain sufficient dosage units, e.g. pills, to last for the specified period of contraception. For example, in a (m, m+4) regimen to be used for the two months August and September, the reservoir must contain 27+26 pills is 53 pills.

“The pill” as used herein means any oral monophasic contraceptive pill.

‘A pill’ as used herein means any discrete dosage unit such as a pill, a tablet, a dragée or a capsule.

A spray device as used herein means any spray device with sufficient active ingredient(s) for at least one cycle of contraception.

A dosage unit useful in the subject invention may comprise an estrogen, a progestogen or combinations thereof. It may optionally also contain other active ingredients such as anti-microbials, folic acid, vitamins etc.

Progestogen as used herein can be any suitable progestogen, such as desogestrel, etonogestrel, levonorgestrel, norgestimate, gestodene, norelgestromin, nomegestrol acetate, dienogest, drospirenone, or any other steroidal or non-steroidal compound with progestogenic activity.

The estrogenic compound as used herein can be any suitable estrogen (or salt thereof or ester thereof), such as estradiol, estriol, mestranol and ethinyl-estradiol or any other steroidal or non-steroidal estrogen with estrogenic activity.

In a specific embodiment of the subject invention, the progestogen is desogestrel or etonogestrel. In another embodiment, the progestogen is nomegestrol acetate.

In one embodiment of the subject invention the estrogen is ethinyl estradiol. In another embodiment, the estrogen is estradiol or an ester thereof or a salt thereof, such as estradiol hemihydrate.

In a specific embodiment, the progestogen is etonogestrel and the estrogen is ethinyl estradiol.

In another specific embodiment, the progestogen is nomegestrol acetate and the estrogen is estradiol or a salt thereof or an ester thereof.

In a specific embodiment, the progestogen is etonogestrel and the estrogen is estradiol or a salt thereof or an ester thereof.

In another specific embodiment, the progestogen is nomegestrol acetate and the estrogen is ethinyl estradiol.

As used herein, both non-hormonal phase’ and ‘hormone-free phase’ is a phase (or period or interval) during a cycle in which no hormones are taken or administered.

The terms ‘non-hormonal phase’ or ‘hormone-free phase’ do not mean or imply that the hormones are not active within the female body.

As used herein, ‘hormonal phase’ is a phase (or period or interval) during a cycle in which hormones are taken/administered.

Thus, the subject invention provides a method of human female contraception which comprises:
(i) administering a monophasic oral contraceptive dosage unit once a day starting on numerical date ‘m+4’ of a month continuously until numerical date ‘m’ of the following month; and
(ii) not administering the monophasic oral contraceptive dosage unit in the numerical dates between ‘m’ and ‘m+4’;
   wherein ‘m’ is a numerical date of a month from 1-24 and wherein the method is repeatedly carried out for at least two cycles.

The subject invention further envisages a method of human female contraception which comprises:
(i) administering a monophasic oral contraceptive dosage unit once a day starting on numerical date ‘y+5’ of a month continuously until numerical date ‘y’ of the following month; and
(ii) not administering the monophasic oral contraceptive dosage unit in the numerical dates between ‘y’ and ‘y+5’;
wherein ‘y’ is a numerical date of a month from 1-23 and wherein the method is repeatedly carried out for at least two cycles.
The subject invention also involves a method of human female contraception which comprises:
(i) administering a monophasic oral contraceptive dosage unit once a day starting on numerical date ‘z+6’ of a month continuously until numerical date ‘z’ of the following month; and
(ii) not administering the monophasic oral contraceptive dosage unit in the numerical dates between ‘z’ and ‘z+6
wherein ‘z’ is a numerical date of a month from 1-22 and wherein the method is repeatedly carried out for at least two cycles.
The subject invention additionally provides a method of human female contraception which comprises:
(i) administering a monophasic oral contraceptive dosage unit once a day starting on numerical date ‘p+7’ of a month continuously until numerical date ‘p’ of the following month; and
(ii) not administering the monophasic oral contraceptive dosage unit in the numerical dates between ‘p’ and ‘p+7
wherein ‘p’ is a numerical date of a month from 1-21 and wherein the method is repeatedly carried out for at least two cycles.

The method of the subject invention can be used for any number of months starting with at least two months, i.e. for two, three, four, five, six, etc. months. In one embodiment, the method is used for at least two months. In a specific embodiment, the method is used for at least three months.

In the methods of contraception of the subject invention, the hormonal-phase between months is not constant. The non-hormonal phase on the other hand is constant between calendar months. In spite thereof, in all embodiments envisaged by the subject invention, ovarian suppression (necessary to achieve contraception) is maintained and in certain cases even improved.

Compliance is thus enabled by the fact that a woman can choose a particular numerical day of the month which she finds an easy number to remember. On this day, the last dosage unit of a cycle will always be administered. Contraceptive efficacy is maintained during the period of contraception independent of the fact that the hormonal phase is not constant between months, whereas the non-hormonal phase is constant between months.

Thus, for example in an (m, m+4) regimen, a woman can choose ‘m’ to be any numerical date between 1-24, independent of which month.

For example, the first of the month is mostly an easy number to remember. In that example, in a (m, m+4) regimen, a woman chooses ‘m’ to be 1, i.e. the first of the month. Then, the first dosage unit of a cycle is started each 5^{th} numerical date of the month (‘m+4’ numerical date of the month). For example, the 5^{th} of January, the 5^{th} of February, the 5^{th} of March etc. etc. The last dosage unit of that cycle is then administered on each first numerical date of the following month, for example, the 1^{st} of February, the 1^{st} of March, the 1^{st} of April, etc. etc. The woman now thus only has to remember the same two numerical dates each month, namely the 1^{st} and the 5^{th} independent of the month. Although not limiting the subject invention thereto, assuming that the first dosage unit of a cycle is started on the fifth numerical date of the month at the same time as the last dosage unit of the former cycle is administered on the first day of the month, the duration of the hormonal phase is:
24 days in February
25 days in February of a leap year
26 days in April, June, September, November
27 days in January, March, May, July, August, October, December

Within the same assumption, the duration of the hormone free phase is constant and lasts 4 days. If the time of the day of the administration of the last dosage unit of a cycle is not the same time of the day as the administration of the first dosage unit of the next cycle, then the hormone free phase can be longer up to a maximum of 5 days when e.g. the woman takes the last dosage unit at 00.01 hours on the first of the month and starts the first dosage unit of the next cycle at 23.59 hours on the fourth day of the month. Thus, in the subject example regimen, the hormone-free phase is between 4-5 days but not longer than 5 days.

For example, when looking at a complete (non-leap) year starting in January and assuming that the first dosage unit of a cycle is administered on the fifth numerical date of each month at the same time as the administration of the last dosage unit of the cycle on the first numerical date of the following month, then this (m, m+4) regimen each month has a hormone free-period of 4 days and hormone administration days as follows:

| Jan | Feb | Mar | Apr | May | June | July | Aug | Sept | Oct | Nov | Dec |
|---|---|---|---|---|---|---|---|---|---|---|---|
| 27 | 24 | 27 | 26 | 27 | 26 | 27 | 27 | 26 | 27 | 26 | 27 |

The concept is similar for the (y, y+5) and (z, z+6) and (p, p+7) regimens. Although not limiting the subject invention thereto, assuming that the first dosage unit of a cycle is administered at the same time as the administration of the last dosage unit of the cycle, then in an (y, y+5) regimen, the hormone free phase is at least 5 days but no longer than 6 days; in a (z, z+6) regimen, the hormone-free phase is at least 6 days but no longer than 7 days; and in a (p, p+7) regimen, the hormone-free phase is at least 7 days but no longer than 8 days.

A regimen of the subject invention has at least two advantages: First of all, compliance is enabled because it is much easier to remember, for a woman using a particular monophasic oral contraceptive, that on a particular day of any month she has to administer the last dosage unit and 4, 5, 6 or 7 days later (depending on the regimen she chooses), resulting also in a fixed numerical date of any month, she has to start the first dosage unit of the next cycle. Secondly, a regimen of the subject invention also maintains or improves the suppression of follicular development due to the longer in-situ period of the dosage form and the shorter hormone-free phase; in other words, a regimen of the subject invention maintains or in certain cases even improves ovarian suppression.

The subject invention also provides for a contraceptive kit for human female contraception which comprises a reservoir containing monophasic oral contraceptive dosage units sufficient for at least any two cycles of contraception, one dosage unit to be administered each day starting on numerical date ‘m+4’ of a month and stopping on numerical date ‘m’ of the following month wherein ‘m’ is a numerical date of a month from 1-24.

The subject invention further envisages a contraceptive kit for human female contraception which comprises a reservoir containing monophasic oral contraceptive dosage units sufficient for at least any two cycles of contraception, one dosage unit to be administered each day starting on numerical date ‘y+5’ of a month and stopping on numerical date ‘y’ of the following month wherein ‘y’ is a numerical date of a month from 1-23.

The subject invention also involves a contraceptive kit for human female contraception which comprises a reservoir containing monophasic oral contraceptive dosage units sufficient for at least any two cycles of contraception, one dosage unit to be administered each day starting on numerical date ‘z+6’ of a month and stopping on numerical date ‘z’ of the following month wherein ‘z’ is a numerical date of a month from 1-22.

The subject invention also provides for a contraceptive kit for human female contraception which comprises a reservoir containing monophasic oral contraceptive dosage units sufficient for at least any two cycles of contraception, one dosage unit to be administered each day starting on numerical date ‘p+7’ of a month and stopping on numerical date ‘p’ of the following month wherein ‘p’ is a numerical date of a month from 1-21.

The subject invention provides for a reminder system for a dosage regimen for a reservoir containing daily monophasic oral contraceptive dosage units comprising choosing one particular numerical date of a month from 1-24, independent of the month, as the numerical date on which administration of a daily dosage unit is always stopped and always starting administration of a dosage unit again four days later.

The subject invention also provides for a reminder system for a dosage regimen for a reservoir containing daily monophasic oral contraceptive dosage units comprising choosing one particular numerical date of a month from 1-23, independent of the month, as the numerical date on which administration of a daily dosage unit is always stopped and always starting administration of a dosage unit again five days later.

The subject invention also envisages a reminder system for a dosage regimen for a reservoir containing daily monophasic oral contraceptive dosage units comprising choosing one particular numerical date of a month from 1-22, independent of the month, as the numerical date on which administration of a daily dosage unit is always stopped and always starting administration of a dosage unit again six days later.

The subject invention further involves a reminder system for a dosage regimen for a reservoir containing daily monophasic oral contraceptive dosage units comprising choosing one particular numerical date of a month from 1-21, independent of the month, as the numerical date on which administration of a daily dosage unit is always stopped and always starting administration of a dosage unit again seven days later.

The subject invention also encompasses a contraceptive regimen for dosage units of the subject invention wherein hormones are administered for a defined duration, characterized in that cycle duration varies such as to correspond with the number of the days of the month in which the cycle was started. The defined duration can be any number of months starting from at least two months, i.e. two, three, four, five, six, etc. months. In one embodiment, the defined duration is two months. In a specific embodiment, the defined duration is three months.

The present invention is further described in the following examples which are not in any way intended to limit the scope of the invention as claimed.

### EXAMPLE 1

### Pharmacodynamic Trial: m, m+4 Wherein n=1

An open-label randomized, comparative pharmacodynamic trial is carried out during the months February, March and April with the commercially available contraceptive pill Marvelon® containing 150 micrograms desogestrel and 30 micrograms ethinyl estradiol in a monthly regimen of the subject invention wherein the first pill of a cycle is taken each 5^{th} of the month (m+4) and the last pill is taken the first (m) of the following month versus the standard 21/7 regimen. This trial is carried out in healthy female volunteers to assess the effects of the oral contraceptive pill in this monthly regimen on ovarian function pharmacodynamics) versus the effects of the oral contraceptive pill on ovarian function in the standard 21/7 regimen.

Forty (40) healthy premenopausal women between 18 and 40 years of age at the time of screening participate in the trial for three treatment cycles.

The women are divided into two groups trial arm A and trial arm B.

Trial arm A uses commercially available strips/blisters of Marvelon® (150 micrograms desogestrel and 30 micrograms ethinyl estradiol) and following the standard regimen wherein the pill is taken for 21 days followed by a 7 day pill-free period.

Trial arm B is provided with a bottle of 77 Marvelon® pills containing 150 micrograms desogestrel and 30 micrograms ethinyl estradiol and uses this contraceptive pill in a regimen of the subject invention wherein the first pill is taken each 5^{th} day of the month and the last pill is taken each first day of the following month.

Three times a week serum estradiol (E2), Progesterone (P), LH and FSH is measured and a transvaginal ultrasound scanning is carried out. A physical and gynecological examination is carried out at screening and at the end of treatment (or at premature discontinuation) and cervical cytology is checked at screening.

### EXAMPLE 2

### Pharmacodynamic Trial y, y+5 Wherein y=1

An open-label randomized, comparative pharmacodynamic trial is carried out essentially as described above in Example 1 in a regimen of the subject invention wherein the first pill of a cycle is taken each 6^{th} of the month (y+5) and then stopped on the first (y) of the following month versus the standard 21/7 regimen. The bottle of pills provided to trial arm B contains 74 pills.

### EXAMPLE 3

### Pharmacodynamic Trial z, z+6 Wherein z=1

An open-label randomized, comparative pharmacodynamic trial is carried out essentially as described above in Example 1 in a regimen of the subject invention wherein the first pill of a cycle is taken on each 7^{th} of the month (z+6) and the last pill of a cycle is taken on the first (z) of the following month versus the standard 21/7 regimen. The bottle of pills provided to trial arm B contains 71 pills.

### EXAMPLE 4

### Pharmacodynamic Trial p, p+7 Wherein p=1

An open-label randomized, comparative pharmacodynamic trial essentially as described above in Example 1 in a regimen of the subject invention wherein the first pill of a cycle is taken on each 8^{th} of the month (p+7) and the last pill of a cycle on the first (p) of the following month versus the standard 21/7 regimen. The bottle of pills provided to trial arm B contains 68 pills.

### EXAMPLE 5

### Exploratory Comparative Trial

An open label, five-arm, randomized, group comparative, multicenter trial with different monthly regimens of the commercially available oral contraceptive pill Marvelon® containing 150 micrograms desogestrel and 30 micrograms ethinyl estradiol is carried out versus the standard 21/7 regimen in healthy female volunteers. Contraceptive efficacy, vaginal bleeding characteristics, safety, compliance, and acceptability of these different monthly regimens is assessed compared to the standard 21/7 regimen.

Five hundred (500) healthy premenopausal women between 18 and 40 years of age at the time of screening participate in the trial for one year, i.e. 13 treatment cycles for the standard 21/7 regimen or 12 months for the monthly regimens of the subject invention.

The women are divided into five (5) groups:

Trial arm A: women are provided with commercially available strips/blisters of Marvelon® (150 micrograms desogestrel and 30 micrograms ethinyl estradiol) are used in a standard regimen: 21 days of pill use, followed by a 7 days pill-free period;

Trial arm B: women are provided with a bottle of 77 Marvelon® pills containing 150 micrograms desogestrel and 30 micrograms ethinyl estradiol to be used in a monthly regimen of the subject invention wherein the first pill of a cycle is taken on the 5^{th} of each month (m+4) and the last pill is taken on the first of the following month (m).

Trial arm C: women are provided with a bottle of 74 Marvelon pills containing 150 micrograms desogestrel and 30 micrograms ethinyl estradiol to be used in a monthly regimen of the subject invention wherein first pill of a cycle is taken on the 6^{th} of each month (y+5), last pill is taken on the first of the following month (y).

Trial arm D: women are provided with a bottle of 71 Marvelon pills containing 150 micrograms desogestrel and 30 micrograms ethinyl estradiol to be used in a monthly regimen of the subject invention wherein the first pill of a cycle is taken on the 7^{th} of each month (z+6) and the last pill is taken on the first of the following month (z).

Trial arm E: women are provided with a bottle of 68 Marvelon pills containing 150 micrograms desogestrel and 30 micrograms ethinyl estradiol to be used in a monthly regimen of the subject invention wherein the first pill of a cycle is taken on the 8^{th} of each month (+7) and the last pill is taken on the first of the following month (p).

Assessments occur at screening (within one month before starting treatment) and at 3, 6, 9 and 12 months or at premature discontinuation. At the screening visit, subjects provide medical and gynecological history and undergo a physical and gynecological examination, including cervical cytology. The physical and gynecological examinations are repeated at the last study visit. In addition, clinical safety laboratory test are performed at screening and at the end of treatment. At all study visits, blood pressure and body weight are measured. A transvaginal ultrasound for assessment of endometrial thickness is performed at screening and repeated after one your. Endometrial biopsies are taken if the double layer endometrial thickness is 10 mm or more. Urinary pregnancy test is performed by the subjects before the start of study treatment, at each study visit and if a pregnancy is suspected during the trial. The occurrence of adverse events and the use of concomitant medication is recorded throughout the trial. Vaginal bleeding patterns and compliance is recorded on diary cards.

### EXAMPLE 6

### Safety and Efficacy Trial m, m+4, Wherein m=1

An open-label two-arm, randomized, group-comparative, multicenter trial is carried out to investigate contraceptive efficacy, vaginal bleeding characteristics, compliance, safety and acceptability with the same oral contraceptive pill as used in the examples above in a regimen of the subject invention wherein the first pill of a cycle is taken on each 5^{th} (m+4) of the month and the last pill is taken on the 1^{st} (m) of the following month versus the standard 21/7 regimen.

Thousand three-hundred and thirty (1330) healthy premenopausal women between 18 and 40 years of age at the time of screening participate in the trial for one year, i.e. 13 treatment cycles for the standard 21/7 regimen or 12 months for the monthly regimen of the subject invention.

The women are divided into two (2) groups:

Trial arm A 330 women are provided with commercially available strips/blisters of Marvelon (150 micrograms desogestrel and 30 micrograms ethinyl estradiol) to be used in a standard regimen of 21 days of pill use, followed by a 7 days pill-free period;

Trial arm B: 1000 women are provided with a bottle of 77 Marvelon® pills containing 150 micrograms desogestrel and 30 micrograms ethinyl estradiol to be used in a monthly regimen of the subject invention wherein the first pill is taken on the 5^{th} of each month (m+4) and the last pill is taken on the first of the following month (m);

Assessments occur at screening (within one month before starting treatment) and at 3, 6, 9 and 12 months or at premature discontinuation. At the screening visit, subjects provide medical and gynecological history and undergo a physical and gynecological examination, including cervical cytology. The physical and gynecological examinations and the cervical cytology is repeated at the last study visit. In addition, clinical safety laboratory test is performed at screening and at the end of treatment. At all study visits, blood pressure and body weight is measured. Urinary pregnancy test is performed by the subjects before the start of study treatment, at each study visit and if a pregnancy is suspected during the trial. The occurrence of adverse events and the use of concomitant medication is recorded throughout the trial. Vaginal bleeding patterns and compliance are recorded on diary cards.

The investigated regimen is found to result in very high compliance in comparison to the standard 21/7 regimen.

### EXAMPLE 7

### Safety and Efficacy Trial y, y+5 Wherein y=1

An open-label two-arm, randomized, group-comparative, multicenter trial essentially as described in Example 6 is carried out to investigate contraceptive efficacy, vaginal bleeding characteristics, compliance, safety and acceptability with the same oral monophasic contraceptive pill as used in Examples 1-5 in a monthly regimen of the subject invention wherein the first pill is taken each 6^{th} (y+5) of the month and stopped the 1^{st} (y) of the following month versus the standard 21/7 regimen. Trial arm B is provided with a bottle of 74 Marvelon® pills containing 150 micrograms desogestrel and 30 micrograms ethinyl estradiol.

The investigated regimen is found to result in very high compliance in comparison to the standard 21/7 regimen.

### EXAMPLE 8

### Safety and Efficacy Trial z, z+6 Wherein z=1

An open-label two-arm, randomized, group-comparative, multicenter trial essentially as described in Example 6 is carried out to investigate contraceptive efficacy, vaginal bleeding characteristics, compliance, safety and acceptability with the same oral monophasic contraceptive pill as used in Examples 1-5 in a monthly regimen of the subject invention wherein the first pill is taken each 7^{th} (z+6) of the month and stopped the 1^{st} (z) of the following month versus the standard 21/7 regimen. Trial arm B is provided with a bottle of 71 Marvelon® pills containing 150 micrograms desogestrel and 30 micrograms ethinyl estradiol.

The investigated regimen is found to result in very high compliance in comparison to the standard 21/7 regimen.

### EXAMPLE 9

### Safety and Efficacy Trial p, p+7 Wherein p=1

An open-label two-arm, randomized, group-comparative, multicenter trial essentially as described in Example 6 is carried out to investigate contraceptive efficacy, vaginal bleeding characteristics, compliance, safety and acceptability with the same oral monophasic contraceptive pill as used in Examples 1-5 in a monthly regimen of the subject invention wherein the first pill is taken each 7^{th} (z+6) of the month and stopped the 1^{st} (z) of the following month versus the standard 21/7 regimen. Trial arm B is provided with a bottle of 68 Marvelon® pills containing 150 micrograms desogestrel and 30 micrograms ethinyl estradiol.

The investigated regimen is found to result in very high compliance in comparison to the standard 21/7 regimen.

1. A method of human female contraception which comprises:
(i) administering a monophasic oral contraceptive dosage unit once a day starting on numerical date ‘m+4’ of a month continuously until numerical date ‘m’ of the following month; and
(ii) not administering the monophasic oral contraceptive dosage unit in the numerical dates between ‘m’ and ‘m+4’;
wherein ‘m’ is a numerical date of a month from 1-24 and wherein the method is repeatedly carried out for at least two cycles.

2. A method of human female contraception which comprises:
(i) administering a monophasic oral contraceptive dosage unit once a day starting on numerical date ‘y+5’ of a month continuously until numerical date ‘5’ of the following month; and
(ii) not administering the monophasic oral contraceptive dosage unit in the numerical dates between ‘y’ and ‘y+5’;
wherein ‘y’ is a numerical date of a month from 1-23 and wherein the method is repeatedly carried out for at least two cycles.

3. A method of human female contraception which comprises:
(i) administering a monophasic oral contraceptive dosage unit once a day starting on numerical date ‘z+6’ of a month continuously until numerical date ‘z’ of the following month; and
(ii) not administering the monophasic oral contraceptive dosage unit in the numerical dates between ‘z’ and ‘z+6
wherein ‘z’ is a numerical date of a month from 1-22 and wherein the method is repeatedly carried out for at least two cycles.

4. A method of human female contraception which comprises:
(i) administering a monophasic oral contraceptive dosage unit once a day starting on numerical date ‘p+7’ of a month continuously until numerical date ‘p’ of the following month; and
(ii) not administering the monophasic oral contraceptive dosage unit in the numerical dates between ‘p’ and ‘p+7
wherein ‘p’ is a numerical date of a month from 1-21 and wherein the method is repeatedly carried out for at least two cycles.

5. The method according to claim 1 wherein the monophasic oral contraceptive dosage unit is a pill.

6. The method according to claim 1 wherein the monophasic oral contraceptive dosage unit is a puff from a spray device.

7. The method according to claim 1 wherein the dosage unit comprises an estrogen and a progestogen.

8. The method according to claim 7 wherein the progestogen is desogestrel or etonogestrel and the estrogen is estradiol or a salt thereof or an ester thereof or the estrogen is ethinyl estradiol.

9. The method according to claim 7 wherein the progestogen is nomegestrol acetate and the estrogen is estradiol or a salt thereof or an ester thereof or the estrogen is ethinyl estradiol.

10. A contraceptive kit for human female contraception which comprises a reservoir containing monophasic oral contraceptive dosage units sufficient for at least any two cycles of contraception, one dosage unit to be administered each day starting on numerical date ‘m+4’ of a month and stopping on numerical date ‘m’ of the following month wherein ‘m’ is a numerical date of a month from 1-24.

11. A contraceptive kit for human female contraception which comprises a reservoir containing monophasic oral contraceptive dosage units sufficient for at least any two cycles of contraception, one dosage unit to be administered each day starting on numerical date ‘y+5’ of a month and stopping on numerical date ‘y’ of the following month wherein ‘y’ is a numerical date of a month from 1-23.

12. A contraceptive kit for human female contraception which comprises a reservoir containing monophasic oral contraceptive dosage units sufficient for at least any two cycles of contraception, one dosage unit to be administered each day starting on numerical date ‘z+6’ of a month and stopping on numerical date ‘z’ of the following month wherein ‘z’ is a numerical date of a month from 1-22.

13. A contraceptive kit for human female contraception which comprises a reservoir containing monophasic oral contraceptive dosage units sufficient for at least any two cycles of contraception, one dosage unit to be administered each day starting on numerical date ‘p+7’ of a month and stopping on numerical date ‘p’ of the following month wherein ‘p’ is a numerical date of a month from 1-21.

14. The kit according to claim 10 wherein the dosage unit is a pill.

15. The kit according to claim 10 wherein the dosage unit is a puff from a spray device.

16. The kit according to claim 10 wherein the dosage unit comprises an estrogen and a progestogen.

17. The kit according to claim 16 wherein the progestogen is desogestrel or etonogestrel and the estrogen is estradiol or a salt thereof or an ester thereof or the estrogen is ethinyl estradiol.

18. The kit according to claim 16 wherein the progestogen is nomegestrol acetate and the estrogen is estradiol or a salt thereof or an ester thereof or the estrogen is ethinyl estradiol.

19. A reminder system for a dosage regimen for a reservoir containing daily monophasic oral contraceptive dosage units comprising choosing one particular numerical date of a month from 1-24, independent of the month, as the numerical date on which administration of a daily dosage unit is always stopped and starting administration of a dosage unit again four days later.

20. A reminder system for a dosage regimen for a reservoir containing daily monophasic oral contraceptive dosage units comprising choosing one particular numerical date of a month from 1-23, independent of the month, as the numerical date on which administration of a daily dosage unit is always stopped and starting administration of a dosage unit again five days later.

21. A reminder system for a dosage regimen for a reservoir containing daily monophasic oral contraceptive dosage units comprising choosing one particular numerical date of a month from 1-22, independent of the month, as the numerical date on which administration of a daily dosage unit is always stopped and starting administration of a dosage unit again six days later.

22. A reminder system for a dosage regimen for a reservoir containing daily monophasic oral contraceptive dosage units comprising choosing one particular numerical date of a month from 1-21, independent of the month, as the numerical date on which administration of a daily dosage unit is always stopped and starting administration of a dosage unit again seven days later.

23. The reminder system according to claim 19 wherein the dosage unit is a pill.

24. The reminder system according to claim 19 wherein the dosage unit is a puff from a spray device.

25. A contraceptive regimen wherein hormones are administered for a defined duration, characterized in that cycle duration varies such as to correspond with the number of the days of the month in which the cycle is started.
